(19) United States
(12) Patent Application Publication
(10) Pub. No.: US 2026/0324945 A1
(43) Pub. Date: Oct. 22, 2026

(54) ENDOSCOPY SYSTEM AND OPERATING METHOD THEREOF

(71) Applicants: Remex Medical Corp., TAICHUNG CITY (TW); Remex Medical USA Inc., Diamond Bar, CA (US)

(72) Inventors: Iou-Sheng CHANG, Taichung City (TW); Guan-Ting LIN, Taichung City (TW); Kuan-Hsun WANG, Taichung City (TW)

(21) Appl. No.: 19/650,292

(22) Filed: Apr. 16, 2026

Related U.S. Application Data
(60) Provisional application No. 63/790,752, filed on Apr. 18, 2025.

(30) Foreign Application Priority Data
Mar. 18, 2026 (TW) 115110233

(51) Int. Cl. A61B 1/00; A61B 1/015; A61B 5/06; A61B 34/20

(52) U.S. Cl. CPC A61B 1/00009; A61B 1/00045; A61B 1/00135; A61B 1/015; A61B 5/067; A61B 34/20; A61B 2034/2072

(57) ABSTRACT

An endoscopy system includes an endoscopy device, a processing device and a display. The endoscopy device includes an endoscope. The endoscope is configured to capture a first image information and includes an inertial measurement unit. The inertial measurement unit is configured to sense a motion information of the endoscope. The processing device is electrically connected to the endoscopy device. The processing device calculates a rotational angle of the endoscope according to the motion information, and processes the first image information and the rotational angle to generate a second image information. The display is electrically connected to the processing device and displays an image corresponding to the second image information. The image displays an orientation indicator, and the orientation indicator has an indicating position corresponding to the rotational angle.

### RELATED APPLICATIONS

This application claims priority to U.S. Provisional Application Ser. No. 63/790,752, filed Apr. 18, 2025; and Taiwan Application Serial Number 115110233, filed Mar. 18, 2026, which are herein incorporated by reference.

### BACKGROUND

### Technical Field

The present invention relates to an endoscopy system and an operating method thereof, and more particularly, to an endoscopy system and an operating method thereof that meet clinical usage habits.

### Description of Related Art

An endoscopy device is a device widely used in industrial or medical fields, which allows users to view narrow positions that cannot be observed with the naked eye, and has a particularly significant impact in medicine. Doctors can insert the endoscope into the human body and observe the internal condition of the human body through the endoscopy device, facilitating further diagnosis and judgment. An endoscopy system including the endoscopy device can display the image from the front end of the endoscope via a display.

In conventional endoscopy systems, the coordinate system of the image orientation is entirely determined by the physical orientation of the sensor: in conventional charge coupled device (CCD) systems, the sensor is fixed in the camera head at the handle end and is mechanically separated from the rotatable tube, so the image can remain horizontal; whereas in disposable complementary metal oxide semiconductor (CMOS) systems, the sensor is packaged at the tip of the tube (Chip-on-the-Tip), and the sensor rotates synchronously with the tube, so the image orientation follows accordingly.

In conventional CCD systems, the sensor and the tube are “separated.” In conventional designs, the endoscope tube and the camera head are connected via a coupler. The mechanical principle is: when the doctor rotates the endoscope tube, the internal cylindrical lens assembly indeed also rotates. However, since the CCD sensor at the rear end is fixed inside the camera head, and the camera head is typically held horizontally by the doctor (with the cable facing downward); the physical result is: as long as the CCD sensor itself does not rotate relative to the ground, no matter how the front-end tube rotates, although the content of the image projected onto the sensor changes, the “up, down, left, right” directions are always determined by the placement angle of the CCD.

In conventional disposable CMOS systems, the sensor and the tube are “integrated”. Conventional disposable endoscopes adopt Chip-on-the-Tip technology to reduce costs and minimize size. The mechanical principle is: the CMOS sensor is packaged at the frontmost end of the tube. This means the sensor and the tube are rigidly connected; and the physical result is: when the doctor rotates the tube, the CMOS sensor also physically rotates along with the tube, so the image on the screen directly flips. This result does not conform to user habits, thereby causing inconvenience in user operation.

In view of this, there is currently a lack of an endoscopy system and an operating method thereof that meet clinical usage habits in the market, so relevant practitioners are all seeking solutions.

### SUMMARY

According to an implementation of a structural aspect of the present invention, provided is an endoscopy system including an endoscopy device, a processing device, and a display. The endoscopy device includes an endoscope. The endoscope is configured to capture a first image information and includes an inertial measurement unit. The inertial measurement unit is configured to sense a motion information of the endoscope. The processing device is electrically connected to the endoscopy device. The processing device receives the motion information and the first image information from the endoscopy device, calculates a rotational angle of the endoscope according to the motion information, and processes the first image information and the rotational angle to generate a second image information. The display is electrically connected to the processing device and displays an image corresponding to the second image information. The image displays an orientation indicator, and the orientation indicator has an indicating position corresponding to the rotational angle.

According to an implementation of a method aspect of the present invention, provided is an operating method of an endoscopy system, including: capturing a first image information by an endoscopy device, and sensing a motion information of an endoscope by an inertial measurement unit of the endoscopy device; receiving the motion information and the first image information from the endoscopy device by a processing device, calculating a rotational angle of the endoscope according to the motion information, and processing the first image information and the rotational angle to generate a second image information; and displaying an image corresponding to the second image information by a display. The image displays an orientation indicator, and the orientation indicator has an indicating position corresponding to the rotational angle.

### BRIEF DESCRIPTION OF THE DRAWINGS

The present disclosure can be more fully understood by reading the following detailed description of the embodiment, with reference made to the accompanying drawings as follows:

FIG. 1 is a schematic diagram illustrating an endoscopy system according to a first embodiment of the present invention.

FIG. 2 is a block diagram illustrating an endoscopy device of the endoscopy system of FIG. 1.

FIG. 3 is a schematic diagram illustrating rotational angles of an endoscope of the present invention being 0 degrees, 45 degrees, and 90 degrees, and their corresponding setups and images.

FIG. 4 is a schematic flowchart illustrating an operating method of an endoscopy system according to a second embodiment of the present invention.

FIG. 5 is a schematic diagram illustrating the endoscopy device of FIG. 1.

FIG. 6 is an exploded view of the endoscopy device of FIG. 5.

FIG. 7 is a partial schematic diagram illustrating a dynamic reference assembly and the endoscope of the endoscopy device of FIG. 5.

FIG. 8 is a cross-sectional view of the endoscopy device of FIG. 5 taken along line 8-8.

FIG. 9 is a cross-sectional view of the endoscopy device of FIG. 5 taken along line 9-9.

FIG. 10A is a cross-sectional view of the endoscopy device of FIG. 5 taken along line 10A-10A.

FIG. 10B is a cross-sectional view of the endoscopy device of FIG. 5 taken along line 10B-10B.

FIG. 11 is a schematic flowchart illustrating an assembly method of an endoscopy device according to a third embodiment of the present invention.

FIG. 12 is a flowchart illustrating one step of the assembly method of the endoscopy device of FIG. 11.

FIG. 13 is a schematic assembly diagram illustrating the step of FIG. 12.

### DETAILED DESCRIPTION

The embodiment will be described with the drawings. For clarity, some practical details will be described below. However, it should be noted that the present disclosure should not be limited by the practical details, that is, in some embodiment, the practical details is unnecessary. In addition, for simplifying the drawings, some conventional structures and elements will be simply illustrated, and repeated elements may be represented by the same labels.

It will be understood that when an element (or device) is referred to as be “connected to” another element, it can be directly connected to the other element, or it can be indirectly connected to the other element, that is, intervening elements may be present. In contrast, when an element is referred to as be “directly connected to” another element, there are no intervening elements present. In addition, the terms first, second, third, etc. are used herein to describe various elements or components, these elements or components should not be limited by these terms. Consequently, a first element or component discussed below could be termed a second element or component.

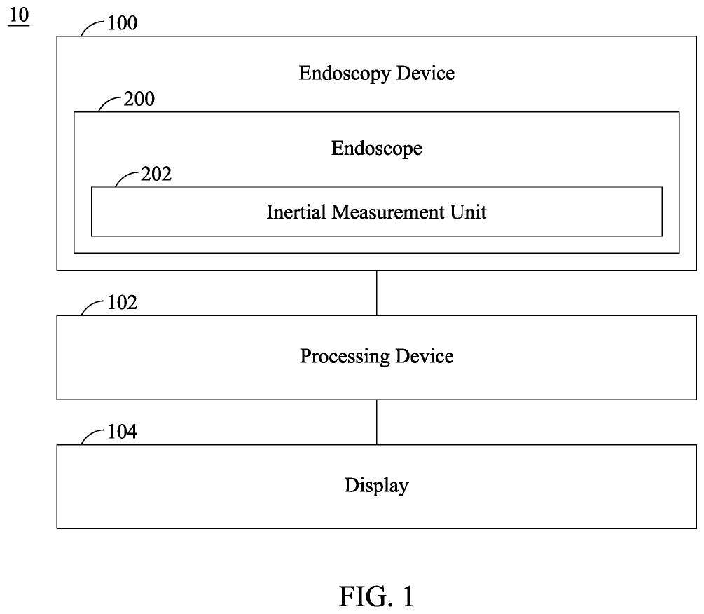

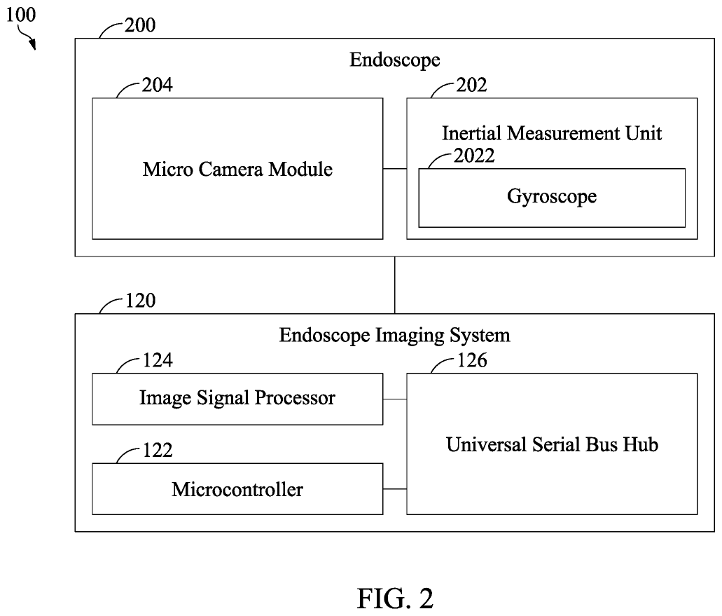

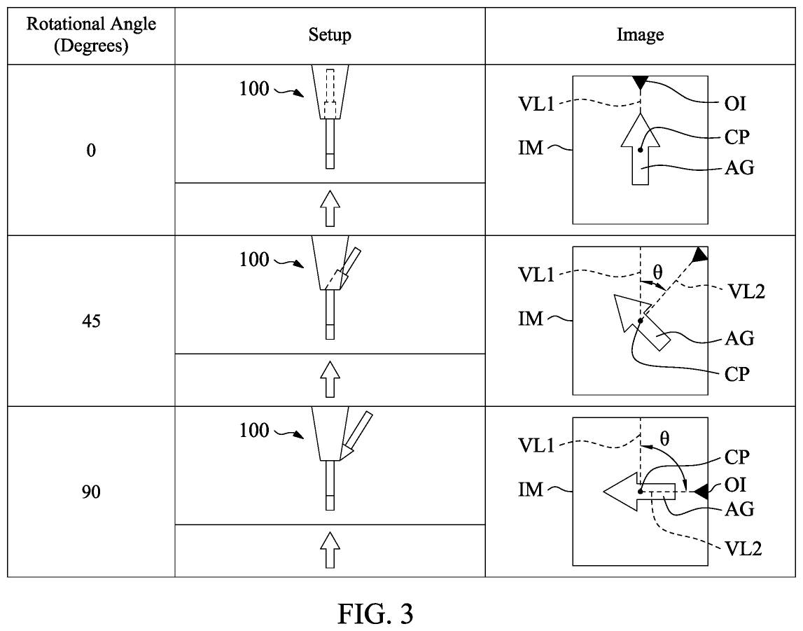

Reference is made to FIG. 1, FIG. 2 and FIG. 3 together, where FIG. 1 is a schematic diagram illustrating an endoscopy system 10 according to a first embodiment of the present invention; FIG. 2 is a block diagram illustrating an endoscopy device 100 of the endoscopy system 10 of FIG. 1; and FIG. 3 is a schematic diagram illustrating rotational angles of an endoscope 200 of the present invention being 0 degrees, 45 degrees, and 90 degrees, and their corresponding setups and images IM. The endoscopy system 10 may be configured corresponding to an optical tracker and includes an endoscopy device 100, a processing device 102, and a display 104. The endoscopy device 100 includes an endoscope 200. The endoscope 200 is configured to capture a first image information and includes an inertial measurement unit (IMU) 202. The inertial measurement unit 202 is configured to sense a motion information of the endoscope 200. The processing device 102 is electrically connected to the endoscopy device 100. The processing device 102 receives the motion information and the first image information from the endoscopy device 100, calculates a rotational angle of the endoscope 200 according to the motion information, and processes the first image information and the rotational angle to generate a second image information. The display 104 is electrically connected to the processing device 102 and displays an image IM corresponding to the second image information. The image IM displays an orientation indicator OI, and the orientation indicator OI has an indicating position corresponding to the rotational angle.

Specifically, the endoscope 200 further includes a micro camera module (MCM) 204, and the micro camera module 204 is electrically connected to the inertial measurement unit 202. The inertial measurement unit 202 includes a gyroscope 2022. The gyroscope 2022 is configured to measure an angular velocity of the endoscope 200, and the motion information includes the angular velocity. The processing device 102 calculates the rotational angle according to the angular velocity. In other words, after raw data acquired by the inertial measurement unit 202 undergoes attitude calculation and dynamic filtering processing, the rotational angle of the endoscope tube of the endoscope 200 can be accurately calculated. Furthermore, the image IM has an image center point CP. When the rotational angle is a first angle value, the indicating position is a first indicating position, and a first virtual line VL1 is formed between the first indicating position and the image center point CP. When the rotational angle is a second angle value, the indicating position is a second indicating position, and a second virtual line VL2 is formed between the second indicating position and the image center point CP. The first angle value is different from the second angle value. An included angle θ between the first virtual line VL1 and the second virtual line VL2 is equal to the rotational angle. The orientation indicator OI can be configured for a user to determine a current rotational condition and a current viewing-angle direction of the endoscope 200. In addition, the endoscopy device 100 further includes an endoscope imaging system 120. The endoscope imaging system 120 is connected to the endoscope 200 and includes a microcontroller 122, an image signal processor 124 and a universal serial bus (USB) hub 126. The microcontroller 122 and the image signal processor 124 are connected to the USB hub 126. The endoscope imaging system 120 is configured to transmit multiple information (e.g., the motion information and the first image information) to the processing device 102.

For example, in FIG. 3, the environmental setup at a first time point is that the endoscope 200 faces downward to capture the first image information, and the first image information includes an arrow graphic AG. When the rotational angle of the endoscope 200 is 0 degrees (the first angle value), the indicating position of the orientation indicator OI in the image IM is at the upper edge of the image, which corresponds to the first indicating position; and the first virtual line VL1 is formed between the first indicating position and the image center point CP.

The environmental setup at a second time point is that a user rotates the endoscope 200 45 degrees clockwise along a horizontal plane, and the endoscope 200 still faces the arrow graphic AG. When the rotational angle is 45 degrees (the second angle value), the indicating position of the orientation indicator OI in the image IM is at the upper right corner edge of the image, which corresponds to the second indicating position; and the second virtual line VL2 is formed between the second indicating position and the image center point CP. The first angle value is different from the second angle value. The included angle θ between the first virtual line VL1 and the second virtual line VL2 is equal to the rotational angle, that is, the included angle θ is equal to 45 degrees.

Similarly, the environmental setup at a third time point is that the user rotates the endoscope 200 90 degrees clockwise along the horizontal plane, and the endoscope 200 still faces the arrow graphic AG. When the rotational angle is 90 degrees, the indicating position of the orientation indicator OI in the image IM is at the right edge of the image, which corresponds to another second indicating position; and the second virtual line VL2 is formed between this another second indicating position and the image center point CP. The included angle θ between the first virtual line VL1 and the second virtual line VL2 is equal to the rotational angle, that is, the included angle θ is equal to 90 degrees. The other rotational angles can be deduced by analogy.

In one embodiment, the orientation indicator OI may be a triangular orientation indicator, which can be red in color and can point inward or outward (the orientation indicator OI in FIG. 3 points inward). When the user rotates the endoscope 200, the orientation indicator OI in the image IM can move synchronously to a corresponding indicating position. However, the present invention is not limited thereto.

Therefore, the endoscopy system 10 according to the present invention utilizes the inertial measurement unit 202 to obtain the rotational angle of the endoscope 200, then through software image processing by the processing device 102, prevents the image IM from rotating, and adds the orientation indicator OI (i.e., the triangular orientation indicator) to the image IM, allowing the user to determine the current rotational condition and facing direction (i.e., the current viewing-angle direction) of the endoscope 200.

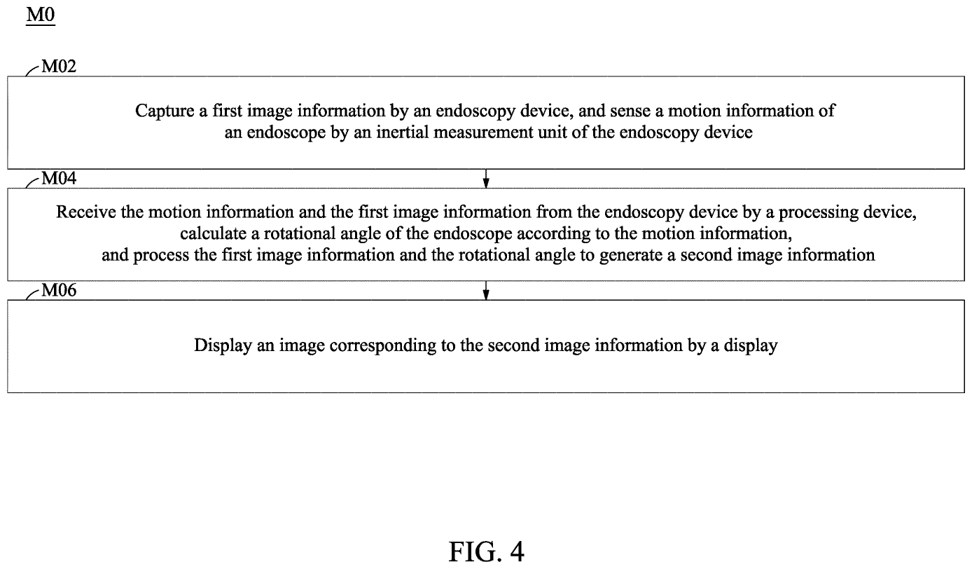

Reference is made to FIG. 1, FIG. 2, FIG. 3 and FIG. 4 together, where FIG. 4 is a schematic flowchart illustrating an operating method M0 of an endoscopy system according to a second embodiment of the present invention. The operating method M0 of the endoscopy system can be applied to the endoscopy system 10 of FIG. 1. The operating method M0 of the endoscopy system includes performing steps M02, M04, M06. Step M02 includes capturing a first image information by an endoscopy device 100, and sensing a motion information of an endoscope 200 by an inertial measurement unit 202 of the endoscopy device 100. Step M04 includes receiving the motion information and the first image information from the endoscopy device 100 by a processing device 102, calculating a rotational angle of the endoscope 200 according to the motion information, and processing the first image information and the rotational angle to generate a second image information. Step M06 includes displaying an image IM corresponding to the second image information by a display 104. The image IM displays an orientation indicator OI, and the orientation indicator OI has an indicating position corresponding to the rotational angle.

Therefore, the operating method M0 of the endoscopy system according to the present invention can prevent the image IM from rotating and add the orientation indicator OI, which quite conforms to user operating habits, thereby solving the problem in the prior art that clinical usage habits cannot be satisfied.

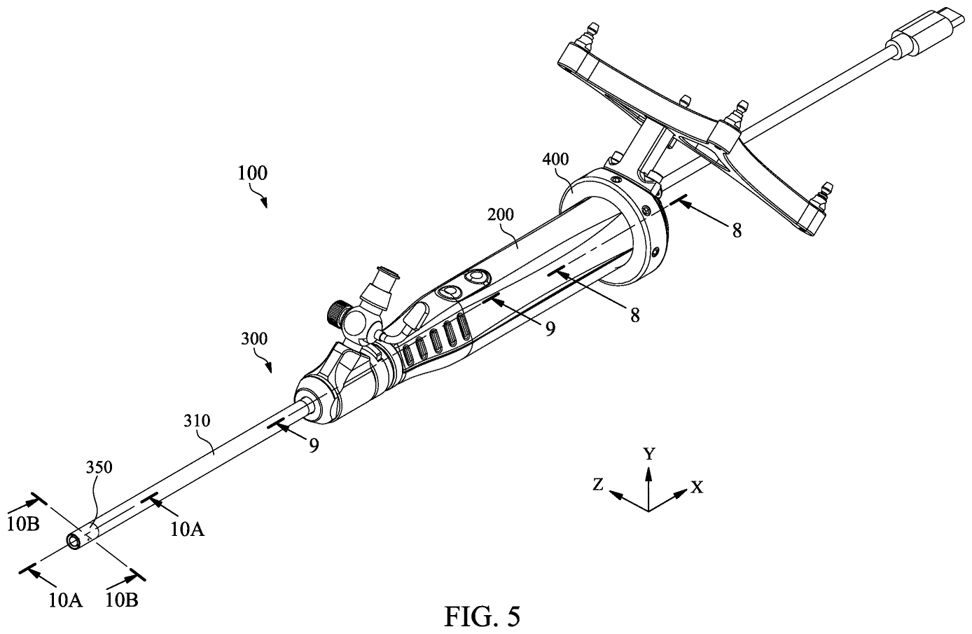

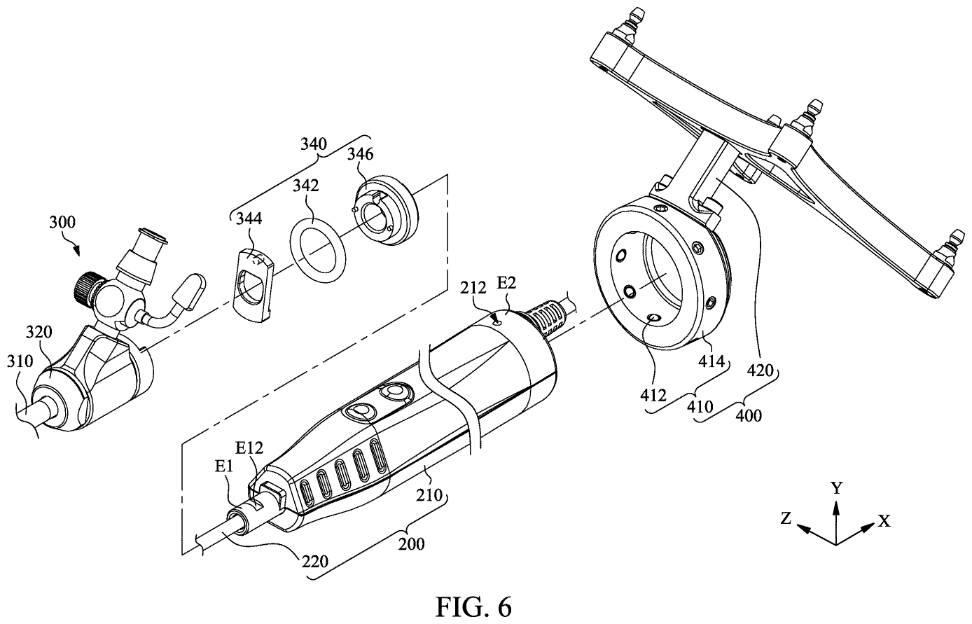

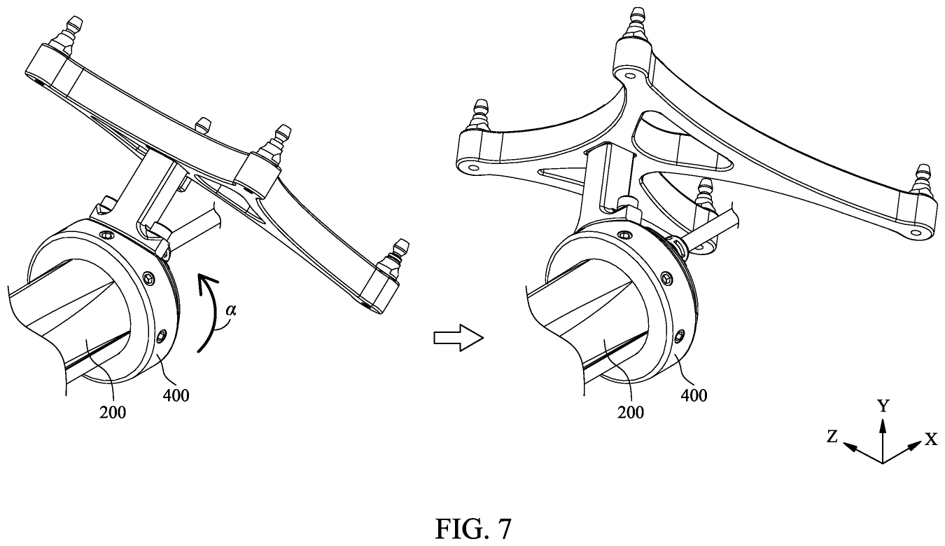

Reference is made to FIG. 1, FIG. 2, FIG. 3, FIG. 5, FIG. 6 and FIG. 7 together, where FIG. 5 is a schematic diagram illustrating the endoscopy device 100 of FIG. 1; FIG. 6 is an exploded view of the endoscopy device 100 of FIG. 5; and FIG. 7 is a partial schematic diagram illustrating a dynamic reference assembly 400 and the endoscope 200 of the endoscopy device 100 of FIG. 5. The endoscopy device 100 can be configured corresponding to an optical tracker. The endoscopy device 100 includes an endoscope 200, a sleeve assembly 300 (a sheath), and a dynamic reference assembly 400. The endoscope 200 has a first end E1 (a front end) and a second end E2 (a rear end), and the first end E1 is opposite to the second end E2. The sleeve assembly 300 is detachably disposed at the first end E1 of the endoscope 200, and the sleeve assembly 300 includes a sleeve 310. The sleeve 310 is straight (the sleeve 310 is a straight tube). The dynamic reference assembly 400 is rotatably positioned at the second end E2 of the endoscope 200, and is configured to be detected by the optical tracker to obtain a position of the endoscope 200. When the sleeve assembly 300 is disposed at the first end E1 of the endoscope 200, the second end E2 of the endoscope 200 is away from the sleeve assembly 300, and the sleeve assembly 300 sleeves the first end E1 of the endoscope 200.

Specifically, the endoscope 200 includes a body 210 and an endoscope tube 220. The body 210 has the first end E1 and the second end E2. The endoscope tube 220 is connected to the first end E1 of the body 210 and is straight (the endoscope tube 220 is a straight tube). The shape of the endoscope tube 220 is the same as (identical to) that of the sleeve 310. When the dynamic reference assembly 400 is positioned at the second end E2 of the endoscope 200, the dynamic reference assembly 400 is detected by the optical tracker to obtain the position of the endoscope 200. The inertial measurement unit 202 is disposed inside the body 210, and the micro camera module 204 is disposed inside a front end of the endoscope tube 220.

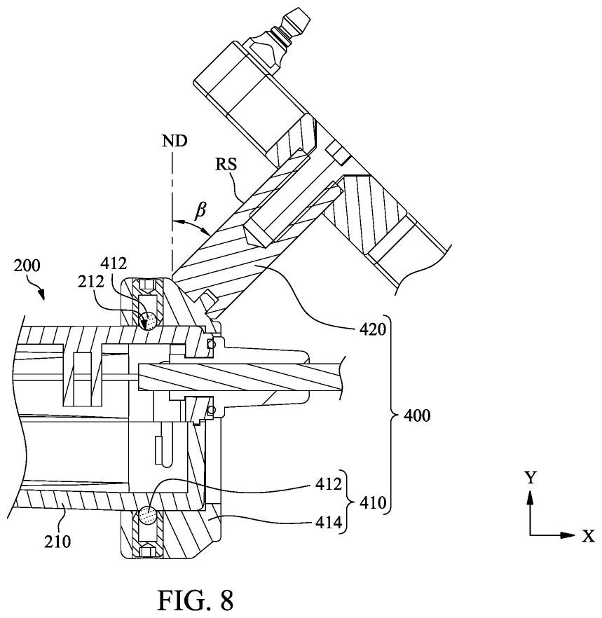

Reference is made to FIG. 5, FIG. 6, FIG. 7 and FIG. 8 together, where FIG. 8 is a cross-sectional view of the endoscopy device 100 of FIG. 5 taken along line 8-8. The dynamic reference assembly 400 includes a rotating ring 410 and a dynamic reference frame (DRF) 420. The rotating ring 410 is rotatable by an angle α along a YZ plane. The dynamic reference frame 420 is connected to the rotating ring 410 and has a reference surface RS. The endoscope 200 has a normal direction ND (same as Y-axis direction). An included angle β is formed between the reference surface RS and the normal direction ND, and the included angle β is greater than 0 degrees and less than 90 degrees (an acute angle). The rotating ring 410 includes at least one positioning bead 412. The second end E2 of the endoscope 200 includes a groove 212. When the dynamic reference assembly 400 is positioned at the second end E2 of the endoscope 200, the at least one positioning bead 412 is correspondingly embedded in the groove 212. Therefore, the present invention utilizes the rotatable dynamic reference assembly 400, allowing the user to adaptively adjust the direction of the dynamic reference frame 420 according to their own usage habits, thereby significantly increasing the convenience of operation.

The number of the at least one positioning bead 412 is plural, and the rotating ring 410 further includes an annular body 414. The positioning beads 412 are spaced apart in the annular body 414, and the dynamic reference frame 420 is fixedly connected to the annular body 414. In one embodiment, the number of the positioning beads 412 is eight, the angle α can be equal to 45 degrees, and the included angle β can be equal to 45 degrees. In other embodiments, the number of the grooves 212 at the second end E2 of the endoscope 200 can be plural (for example, the number of the grooves 212 can be two, and the two grooves 212 can be respectively disposed on opposite two sides of the second end E2). The more the number of the grooves 212 is, the better the engagement effect between the second end E2 and the rotating ring 410 is. However, the present invention is not limited thereto.

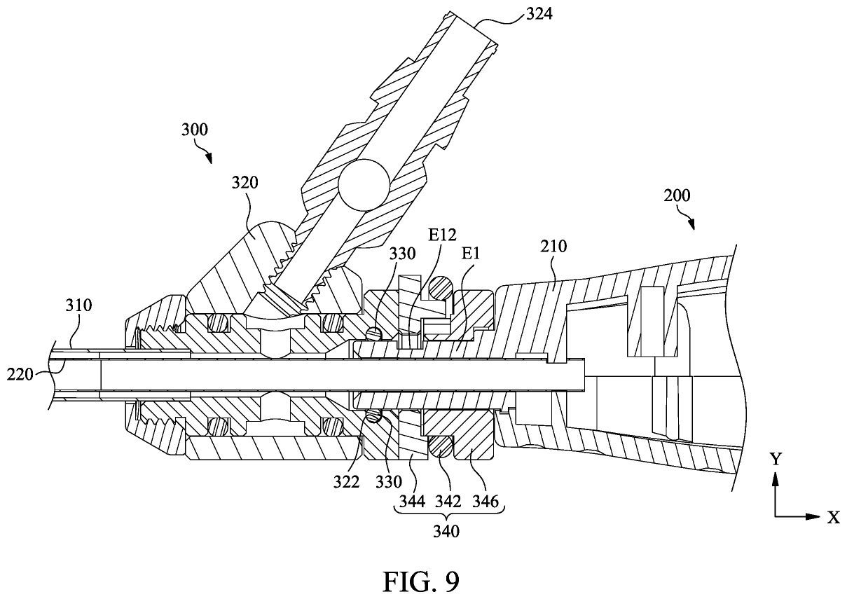

Reference is made to FIG. 5, FIG. 6 and FIG. 9 together, where FIG. 9 is a cross-sectional view of the endoscopy device 100 of FIG. 5 taken along line 9-9. The sleeve assembly 300 of the endoscopy device 100 further includes a sleeve attachment portion 320, a sealing ring 330, and an elastic limiting assembly 340. The sleeve attachment portion 320 is connected to the sleeve 310 and has an inner sidewall 322 and a water inlet 324. The sealing ring 330 is embedded in the inner sidewall 322. When the sleeve assembly 300 is disposed at the first end E1 of the endoscope 200, the sealing ring 330 seals a gap between the sleeve attachment portion 320 and the first end E1 of the endoscope 200. Furthermore, the first end E1 of the endoscope 200 includes a recess E12. The elastic limiting assembly 340 is connected to the sleeve attachment portion 320 and includes an elastic member 342, a pressing plate 344, and a connection portion 346. The elastic member 342 is annular. The pressing plate 344 is connected to the elastic member 342 and is movable between a first position and a second position. The connection portion 346 is connected to the pressing plate 344 and the first end E1. The pressing plate 344 is limited by the elastic member 342, so that the pressing plate 344 at the first position engages with the recess E12. In one embodiment, the sealing ring 330 may be an O-ring structure. Thus, the present invention utilizes the sealing ring 330 to seal an opening of the sleeve assembly 300 to prevent water leakage from the inside.

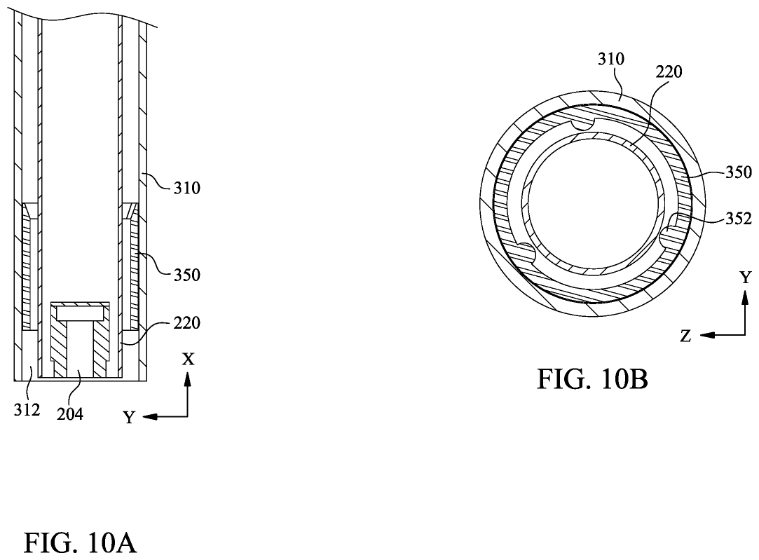

Reference is made to FIG. 5, FIG. 6 and FIG. 10A together, where FIG. 10A is a cross-sectional view of the endoscopy device 100 of FIG. 5 taken along line 10A-10A. The micro camera module 204 is disposed inside the front end of the endoscope tube 220 of the endoscope 200, and the micro camera module 204 is configured to capture the image in front of the endoscope tube 220. The sleeve assembly 300 further includes a liner sleeve 350. The liner sleeve 350 is disposed in an internal space of the sleeve 310, and the liner sleeve 350 is configured to limit the endoscope tube 220. The liner sleeve 350 is close to a front end of the sleeve 310. In one embodiment, the endoscope 200 can be a disposable endoscope, and the micro camera module 204 can use a complementary metal oxide semiconductor (CMOS) photosensitive element. However, the present invention is not limited thereto.

Reference is made to FIG. 5, FIG. 9, FIG. 10A and FIG. 10B together, where FIG. 10B is a cross-sectional view of the endoscopy device 100 of FIG. 5 taken along line 10B-10B. The liner sleeve 350 includes a plurality of protrusions 352. The protrusions 352 protrude toward an interior of the liner sleeve 350 and are configured to abut against an outer wall of the endoscope tube 220 to limit the endoscope tube 220. A water outlet area is formed between the liner sleeve 350 and the endoscope tube 220. Water can enter from the water inlet 324 of the sleeve attachment portion 320, then pass through the water outlet area, and finally flow out from a front water outlet 312 of the sleeve 310. In the present embodiment, the front ends of the endoscope tube 220 and the sleeve 310 are flat ends; and in other embodiments, the front ends of the endoscope tube 220 and the sleeve 310 may be beveled ends, but the present invention is not limited thereto. Thus, the present invention utilizes the protrusions 352 of the liner sleeve 350 to center and position the endoscope tube 220 at the front end of the sleeve 310, thereby increasing the operational stability of the endoscopy device 100.

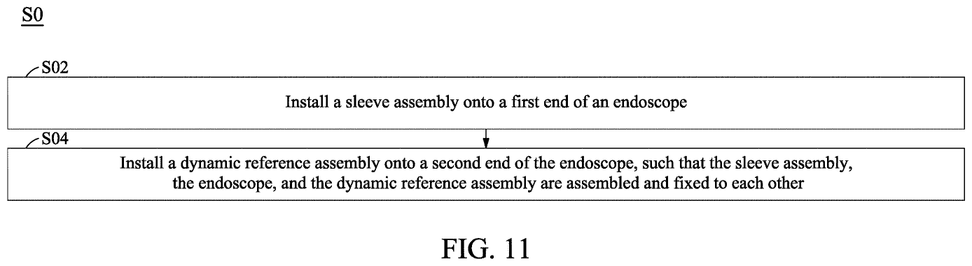

Reference is made to FIG. 5, FIG. 6 and FIG. 11 together, where FIG. 11 is a schematic flowchart illustrating an assembly method S0 of an endoscopy device according to a third embodiment of the present invention. The assembly method S0 of the endoscopy device can be applied to the endoscopy device 100 of FIG. 5. The assembly method S0 of the endoscopy device includes performing steps S02, S04. Step S02 includes installing a sleeve assembly 300 onto a first end E1 of an endoscope 200. Step S04 includes installing a dynamic reference assembly 400 onto a second end E2 of the endoscope 200, such that the sleeve assembly 300, the endoscope 200, and the dynamic reference assembly 400 are assembled and fixed to each other. The endoscopy device 100 is configured corresponding to an optical tracker. The first end E1 is opposite to the second end E2. The sleeve assembly 300 is detachably disposed at the first end E1 of the endoscope 200. The sleeve assembly 300 includes a sleeve 310. The sleeve 310 is straight (the sleeve 310 is a straight tube). The second end E2 of the endoscope 200 is away from the sleeve assembly 300, and the sleeve assembly 300 sleeves the first end E1 of the endoscope 200. The dynamic reference assembly 400 is rotatably positioned at the second end E2 of the endoscope 200, and is configured to be detected by the optical tracker to obtain a position of the endoscope 200. In other embodiments, step S04 may be performed before step S02, and the present invention is not limited thereto.

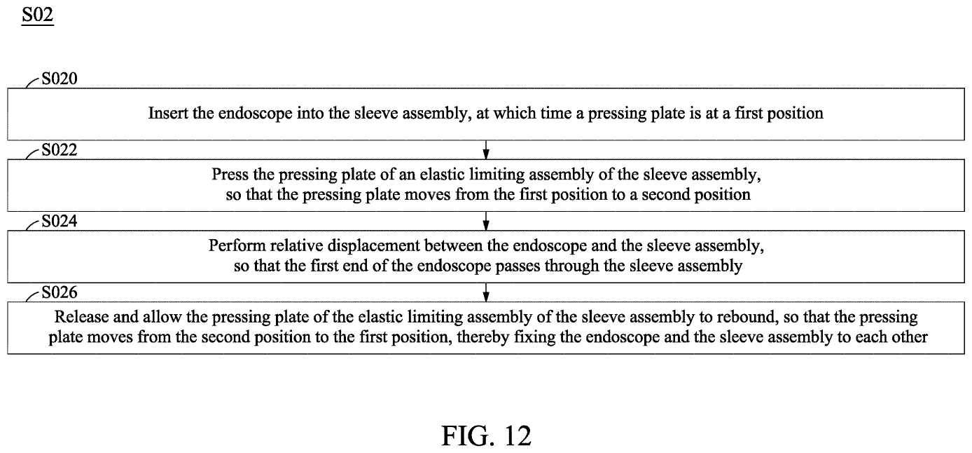

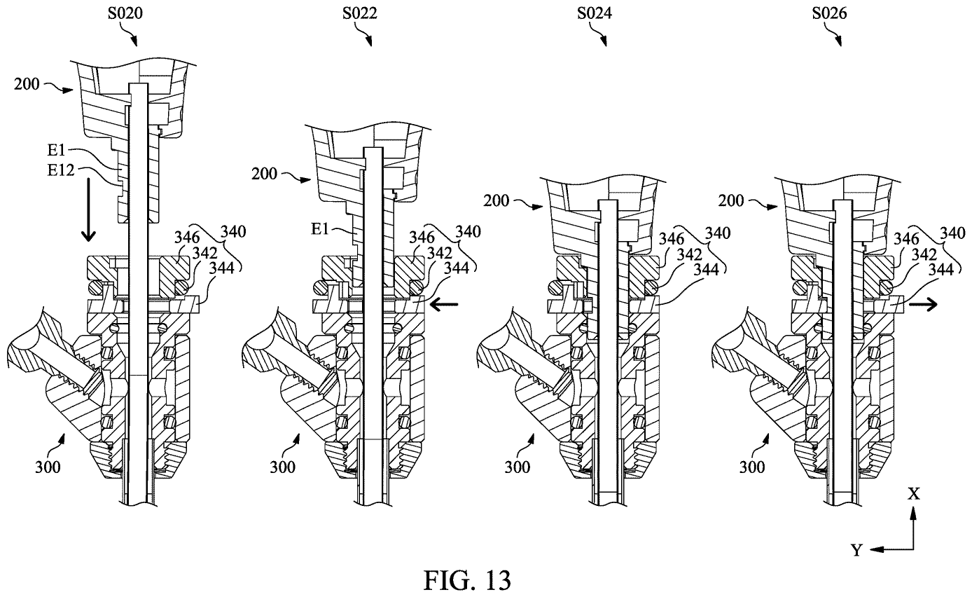

Reference is made to FIG. 5, FIG. 6, FIG. 9, FIG. 11, FIG. 12 and FIG. 13 together, where FIG. 12 is a flowchart illustrating one step S02 of the assembly method S0 of the endoscopy device of FIG. 11; and FIG. 13 is a schematic assembly diagram illustrating step S02 of FIG. 12. Step S02 may include performing steps S020, S022, S024, S026. Step S020 includes inserting the endoscope 200 into the sleeve assembly 300, at which time the pressing plate 344 is at a first position, and the first end E1 of the endoscope 200 is blocked by the pressing plate 344 and cannot be fully inserted into the sleeve assembly 300. Step S022 includes pressing the pressing plate 344 of an elastic limiting assembly 340 of the sleeve assembly 300 (e.g., a user presses the pressing plate 344 by hand), so that the pressing plate 344 moves from the first position to a second position. At this time, the pressing plate 344 is at the second position, and the first end E1 of the endoscope 200 can be fully inserted into the sleeve assembly 300. Step S024 includes performing relative displacement between the endoscope 200 and the sleeve assembly 300 (inserting the endoscope 200 to engage with the sleeve assembly 300), so that the first end E1 of the endoscope 200 passes through the sleeve assembly 300. At this time, the pressing plate 344 remains pressed and is at the second position. Step S026 includes releasing and allowing the pressing plate 344 of the elastic limiting assembly 340 of the sleeve assembly 300 to rebound, so that the pressing plate 344 moves from the second position to the first position, thereby fixing the endoscope 200 and the sleeve assembly 300 to each other. The first end E1 of the endoscope 200 includes a recess E12. When the pressing plate 344 moves from the second position to the first position (released and rebounded), the pressing plate 344 at the first position is engaged with the recess E12.

In other embodiments, the endoscope tube and the sleeve of the endoscopy device may be curved tubes, that is, the endoscope tube and the sleeve are curved. The assembly method S0 of the endoscopy device and its step S02 in FIG. 12 and FIG. 13 can be applied to a curved-tube type endoscopy device, and the present invention is not limited thereto.

As can be seen from the above implementations, the present invention has the following advantages: First, the image rotation problem of the endoscope using the “sensor and tube are integrated, Chip-on-the-Tip” approach is solved, so that the present invention can match the conventional image presentation habit of “sensor and tube are separated”. The endoscopy system utilizes the inertial measurement unit to obtain the rotational angle of the endoscope, then through software image processing, prevents the image from rotating, and adds the orientation indicator to the image, allowing the user to determine the current rotational condition and facing direction of the endoscope. Second, by employing the rotatable dynamic reference assembly, users can adaptively adjust the direction of the dynamic reference frame according to their own usage habits, thereby significantly enhancing operational convenience. Third, by using the protrusions of the liner sleeve, the endoscope tube can be centered and positioned at the front end of the sleeve, thereby increasing the operational stability of the endoscopy device. Fourth, by using the sealing ring to seal the opening of the sleeve assembly, water leakage from the inside is prevented.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An endoscopy system, comprising:
an endoscopy device, comprising an endoscope that is configured to capture a first image information and comprises an inertial measurement unit configured to sense a motion information of the endoscope;
a processing device, electrically connected to the endoscopy device, the processing device receiving the motion information and the first image information from the endoscopy device, calculating a rotational angle of the endoscope according to the motion information, and processing the first image information and the rotational angle to generate a second image information; and
a display, electrically connected to the processing device, and displaying an image corresponding to the second image information;
wherein the image displays an orientation indicator, and the orientation indicator has an indicating position corresponding to the rotational angle.

2. The endoscopy system according to claim 1, wherein the inertial measurement unit comprises:
a gyroscope, configured to measure an angular velocity of the endoscope, the motion information comprising the angular velocity;
wherein the processing device calculates the rotational angle according to the angular velocity.

3. The endoscopy system according to claim 1, wherein,
the image has an image center point;
when the rotational angle is a first angle value, the indicating position is a first indicating position, and a first virtual line is formed between the first indicating position and the image center point;
when the rotational angle is a second angle value, the indicating position is a second indicating position, and a second virtual line is formed between the second indicating position and the image center point, and the first angle value is different from the second angle value;
an included angle between the first virtual line and the second virtual line is equal to the rotational angle; and
the orientation indicator is configured for a user to determine a current rotational condition and a current viewing-angle direction of the endoscope.

4. The endoscopy system according to claim 1, wherein the endoscope has a first end and a second end, the first end is opposite to the second end; and the endoscopy device further comprises:
a sleeve assembly, detachably disposed at the first end of the endoscope, the sleeve assembly comprising a sleeve, the sleeve being curved or straight; and
a dynamic reference assembly, rotatably positioned at the second end of the endoscope and configured to be detected by an optical tracker to obtain a position of the endoscope;
wherein when the sleeve assembly is disposed at the first end of the endoscope, the second end of the endoscope is away from the sleeve assembly, and the sleeve assembly sleeves the first end of the endoscope.

5. The endoscopy system according to claim 4, wherein,
the endoscope further comprises:
a body, having the first end and the second end; and
an endoscope tube, connected to the first end of the body, and having a shape being identical to that of the sleeve; and
the sleeve assembly further comprises:
a liner sleeve, disposed in an internal space of the sleeve, the liner sleeve being configured to limit the endoscope tube.

6. The endoscopy system according to claim 5, wherein the liner sleeve comprises:
a plurality of protrusions, protruding toward an interior of the liner sleeve and configured to abut against an outer wall of the endoscope tube to limit the endoscope tube;
wherein a water outlet area is formed between the liner sleeve and the endoscope tube.

7. The endoscopy system according to claim 4, wherein the dynamic reference assembly comprises:
a rotating ring, comprising at least one positioning bead; and
a dynamic reference frame, connected to the rotating ring and having a reference surface, the endoscope having a normal direction, an included angle being formed between the reference surface and the normal direction, the included angle being greater than 0 degrees and less than 90 degrees;
wherein the second end of the endoscope comprises a groove, and when the dynamic reference assembly is positioned at the second end of the endoscope, the at least one positioning bead is correspondingly embedded in the groove.

8. The endoscopy system according to claim 7, wherein a number of the at least one positioning bead is plural, and the rotating ring further comprises:
an annular body, the plurality of positioning beads being spaced apart in the annular body, and the dynamic reference frame being fixedly connected to the annular body.

9. The endoscopy system according to claim 4, wherein the sleeve assembly further comprises:
a sleeve attachment portion, connected to the sleeve and having an inner sidewall; and
a sealing ring, embedded in the inner sidewall;
wherein when the sleeve assembly is disposed at the first end of the endoscope, the sealing ring seals a gap between the sleeve attachment portion and the first end of the endoscope.

10. The endoscopy system according to claim 9, wherein the first end of the endoscope comprises a recess, and the sleeve assembly further comprises:
an elastic limiting assembly, connected to the sleeve attachment portion and comprising:
an elastic member; and
a pressing plate, connected to the elastic member and movable between a first position and a second position;
wherein the pressing plate is limited by the elastic member, so that the pressing plate at the first position is engaged with the recess.

11. An operating method of an endoscopy system, comprising:
capturing a first image information by an endoscopy device, and sensing a motion information of an endoscope by an inertial measurement unit of the endoscopy device;
receiving the motion information and the first image information from the endoscopy device by a processing device, calculating a rotational angle of the endoscope according to the motion information, and processing the first image information and the rotational angle to generate a second image information; and
displaying an image corresponding to the second image information by a display;
wherein the image displays an orientation indicator, and the orientation indicator has an indicating position corresponding to the rotational angle.

12. The operating method of the endoscopy system according to claim 11, wherein the inertial measurement unit comprises:
a gyroscope, configured to measure an angular velocity of the endoscope, the motion information comprising the angular velocity;
wherein the processing device calculates the rotational angle according to the angular velocity.

13. The operating method of the endoscopy system according to claim 11, wherein,
the image has an image center point;
when the rotational angle is a first angle value, the indicating position is a first indicating position, and a first virtual line is formed between the first indicating position and the image center point;
when the rotational angle is a second angle value, the indicating position is a second indicating position, and a second virtual line is formed between the second indicating position and the image center point, and the first angle value is different from the second angle value;
an included angle between the first virtual line and the second virtual line is equal to the rotational angle; and
the orientation indicator is configured for a user to determine a current rotational condition and a current viewing-angle direction of the endoscope.

14. The operating method of the endoscopy system according to claim 11, wherein the endoscope has a first end and a second end, the first end is opposite to the second end; and the endoscopy device further comprises:
a sleeve assembly, detachably disposed at the first end of the endoscope, the sleeve assembly comprising a sleeve, the sleeve being curved or straight; and
a dynamic reference assembly, rotatably positioned at the second end of the endoscope and configured to be detected by an optical tracker to obtain a position of the endoscope;
wherein when the sleeve assembly is disposed at the first end of the endoscope, the second end of the endoscope is away from the sleeve assembly, and the sleeve assembly sleeves the first end of the endoscope.

15. The operating method of the endoscopy system according to claim 14, wherein,
the endoscope further comprises:
a body, having the first end and the second end; and
an endoscope tube, connected to the first end of the body, and having a shape being identical to that of the sleeve; and
the sleeve assembly further comprises:
a liner sleeve, disposed in an internal space of the sleeve, the liner sleeve being configured to limit the endoscope tube.

16. The operating method of the endoscopy system according to claim 15, wherein the liner sleeve comprises:
a plurality of protrusions, protruding toward an interior of the liner sleeve and configured to abut against an outer wall of the endoscope tube to limit the endoscope tube;
wherein a water outlet area is formed between the liner sleeve and the endoscope tube.

17. The operating method of the endoscopy system according to claim 14, wherein the dynamic reference assembly comprises:
a rotating ring, comprising at least one positioning bead; and
a dynamic reference frame, connected to the rotating ring and having a reference surface, the endoscope having a normal direction, an included angle being formed between the reference surface and the normal direction, the included angle being greater than 0 degrees and less than 90 degrees;
wherein the second end of the endoscope comprises a groove, and when the dynamic reference assembly is positioned at the second end of the endoscope, the at least one positioning bead is correspondingly embedded in the groove.

18. The operating method of the endoscopy system according to claim 17, wherein a number of the at least one positioning bead is plural, and the rotating ring further comprises:
an annular body, the plurality of positioning beads being spaced apart in the annular body, and the dynamic reference frame being fixedly connected to the annular body.

19. The operating method of the endoscopy system according to claim 14, wherein the sleeve assembly further comprises:
a sleeve attachment portion, connected to the sleeve and having an inner sidewall; and
a sealing ring, embedded in the inner sidewall;
wherein when the sleeve assembly is disposed at the first end of the endoscope, the sealing ring seals a gap between the sleeve attachment portion and the first end of the endoscope.

20. The operating method of the endoscopy system according to claim 19, wherein the first end of the endoscope comprises a recess, and the sleeve assembly further comprises:
an elastic limiting assembly, connected to the sleeve attachment portion and comprising:
an elastic member; and
a pressing plate, connected to the elastic member and movable between a first position and a second position;
wherein the pressing plate is limited by the elastic member, so that the pressing plate at the first position is engaged with the recess.
